(19) United States
(12) Patent Application Publication
(10) Pub. No.: US 2006/0020176 A1
(43) Pub. Date: Jan. 26, 2006

(54) Portable handheld medical diagnostic tool ''Camcorder handle''

(76) Inventor: Jonathan Berall, Brooklyn, NY (US)

Correspondence Address: Jonathan Berall, 173 Columbia Hts, Brooklyn, NY, 11201, US

(21) Appl. No.: 11/185,770

(22) Filed: Jul. 21, 2005

Related U.S. Application Data
(60) Provisional application No. 60/590,139, filed on Jul. 21, 2004.

(51) Int. Cl. A61B 5/00

(52) U.S. Cl. 600/300

(57) ABSTRACT

The portable handheld medical diagnostic tool incorporates camcorder technology and combines multiple diagnostic attachments commonly use by physicians and medical personnel. When attached with various portable instruments, it can be a powerful tool for teaching, diagnostics, therapy of illnesses by virtue of real-time video, audio and other sensory feed on the portable handheld medical diagnostic tool. Video, audio and sensory data can then be transfer to computers or other electronic systems for medical presentation and training or for medical diagnostics. This ability is presently not available.

### BACKGROUND

At present there is no single diagnostic tool that can be able to perform a multitude of tasks. Physicians need a variety of different equipment to perform diagnostics of their patient's health.

Physicians use equipment and tools that are able to provide visual and audio information to diagnose patients. Examples of some of these equipments physician's use are the otoscope for ear examination, and the ophthalmoscope for eye examination. Another example would be laryngoscope for intubation.

The functionality of these standard instruments lacks the ability to record, store, process and transfer information regarding the examination undertaken. Physicians are unable to evaluate the progression of illnesses, devise and evaluate treatment regimes and consult specialists in the field easily.

Discussions:

At present the ability to record real-time information on what is being seen by a physician is not part of the standard medical instrument. Physicians current rely on written observation of their patient's examination. This is a major limitation in diagnostics, therapeutic and teaching as the information physicians see in their exams cannot be easily recorded, stored, processed and transmitted.

The invention is a portable handheld medical diagnostic tool (PHMDT) for physicians. The PHMDT has two major components. The first component is the handle (FIG. 1) and the second component is the attachments of the multitudes of instruments for various diagnostics.

The portable handheld medical diagnostic tool has a number of functionalities that is a result of the variety of attachments. An example of a possible attachment could be related to the intubation. Currently, the standard laryngoscope is used for looking into the larynx to insert a plastic tube into the windpipe to facilitate breathing; intubations. The attachments for a video modified laryngoscope would comprise the blade, light source, camera and handle connectors. All information from the video modified laryngoscope attachment blade would be transferred to the portable handheld medical diagnostic tool using the common attachment connector and be display on the screen. The information can then be stored, processed, and transferred using the transfer communication system. This would improve the success rate of intubations by presenting visual data that is not otherwise available due to the limitation of the current laryngoscope and would facilitate consultation, evaluation, education and training.

Other examples of such attachments would be the video modified otoscopy for the inspection of the ear and the video modified ophthalmoscope for eye examination. Data would then be recorded on the patient's examination that can be used for diagnosis and follow-up evaluation of illnesses and treatments.

### BRIEF DESCRIPTION OF THE DRAWINGS

FIG. 1, is an illustration of the front view of the embodiment.

FIG. 2, is an illustration of the top view of the embodiment.

FIG. 3, is an illustration of the side view of the embodiment.

### DESCRIPTION OF PREFERRED EMBODIMENT

In the figures of the PHMDT, there contains a battery system 1, microprocessor 2, digital display screen 3, transfer communication system connectors 4-6, data storage device 7 and common attachment connectors 8 as the main components.

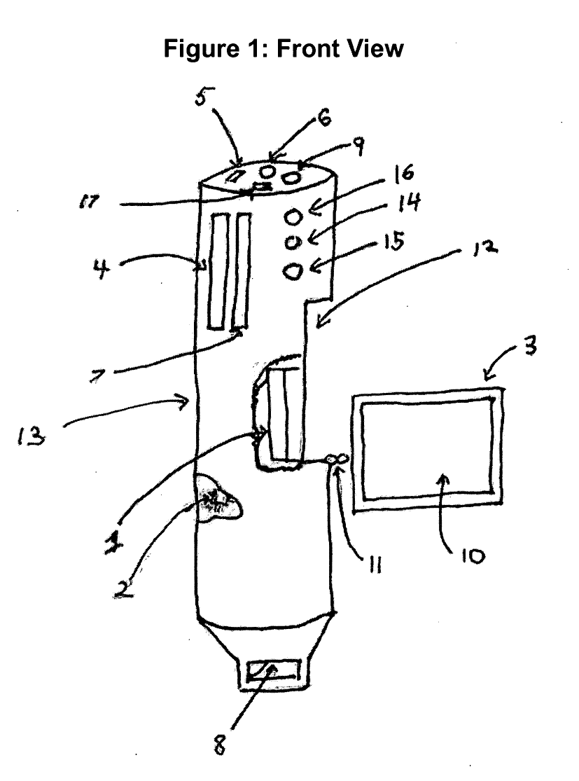

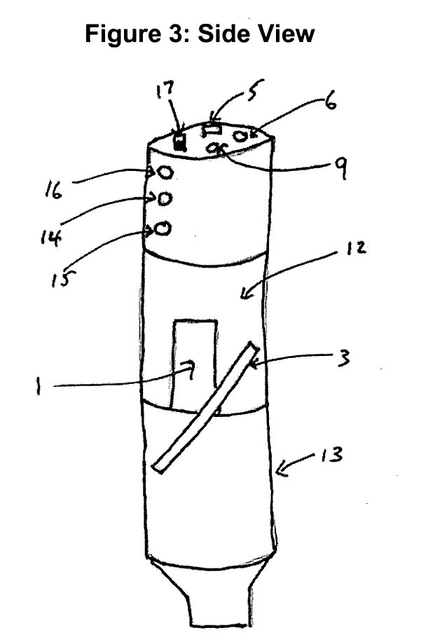

In the battery system 1 (FIG. 1 & FIG. 3), there is a built-in rechargeable battery connector 9 (FIG. 1-3) that can recharge or will be powered using an AC outlet. The battery is use to power the PHMDT and the multitude of attachments.

The microprocessor 2 (FIG. 1) that is in the PHMDT is required to received, process, transmit data and control all components of the PHMDT.

The digital display screen 3 (FIG. 1-3) will display real-time video, audio and sensory information that is coming from the attachments. The digital display screen can also display patient information when synchronized with the hospital database using the wireless communication system 4 (FIG. 1) that is build into the portable handheld medical diagnostic tool. The digital display screen 3 (FIG. 1-3) will present diagnostic information that is received from the attachments. The digital display screen 10 (FIG. 1) utilizes touch panel technology allowing modification of presented data.

The portable handheld medical diagnostic has built-in compact data storage device 7 (FIG. 1). The digital data stored in the memory system 7 (FIG. 1) can be transferred, modified and shared in a presentable way using the transfer communication system 4-6 (FIG. 1-3) on the PHMDT.

There are several kinds of transfer communication system 4-6 (FIG. 1-3) built-in with the PHMDT. The major transfer communication system will be the wireless system 4 (FIG. 1). With the built-in wireless system 4 (FIG. 1), data and information can be stored, exchanged using the network system that is available in the facility. Other transfer communication system could be the utilization of connectivity technology such as USB 5 (FIG. 1-3), PS/2 and other relevant connectivity technologies 6 (FIG. 1-3).

The attachments consist of multitude of functioning tools that would perform a variety of operations and transfers visual, audio and sensory information to the PHMDT. Each attachment will have a common connector 8 (FIG. 1) to the PHMDT. The connectors are based on connectivity technology such as USB, PS/2, infrared and any other relevant connectivity technology that can be incorporated into the PHMDT and its attachments. The attachments are powered by the PHMDT using the common connectors 8 (FIG. 1).

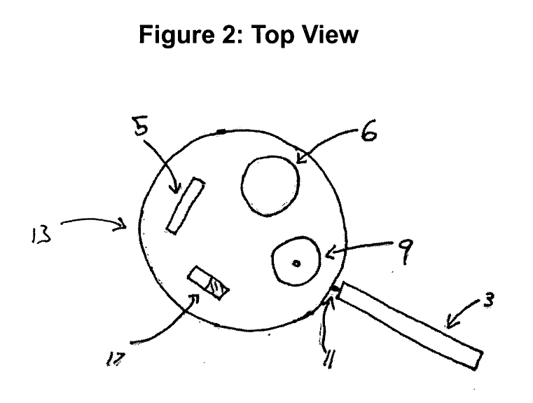

A preferred design on the digital display screen will incorporate polyaxial hinges 11 (FIG. 1-2) mounted onto a cut away slot 12 (FIG. 1 & 3) of the shell 13 (FIG. 1-3) to enable flipping and folding of the display. The polyaxial hinges 11 (FIG. 1-2) can enable freedom of movement.

The PHMDT will incorporate such materials with desired properties and functionality to create the shell 13 (FIG. 1-3). These materials are able to be sterilized and/or protected from contamination. The same materials can also be used to create the structure of the attachments.

There are three LED indicators 14-16 (FIG. 1 & 3) showing PHMDT operations. The first LED 14 (FIG. 1 & 3) indicates the PHMDT being on or off, which can be turned on using the PHMDT switch 17 (FIG. 1 & 3). The second LED 15 (FIG. 1 & 3) indicates low battery and battery charging. Third LED 16 (FIG. 1 & 3) indicates the attachments to the PHMDT.

### SUMMARY OF INVENTION

At present time, there is no technology in the medical application industry that allows physicians to visualize, store, record, process, and transmit their findings on a variety of physical examinations, such as examination of the ear, eye, and placement of breathing tube. Such technology would dramatically improve diagnosis and therapeutic procedures for patients.

This invention has two main components. The first component is to combine digital display technology, communication technology, connectivity technology, battery technology and structure design technology to create a portable handheld medical diagnostic tool. The second components are the attachments that would gather audio, visual and sensory inputs. An example would be a video modified portable laryngoscope, video modified portable otoscope and video modified portable ophthalmoscope.

The benefits of these combinations of technologies are improvements to existing instrument to record, store, process, and transfer information to facilitate consultations, evaluations, education and training.

### REFERENCES

U.S. Pat. No. 5,827,178 October, 1998 Berall

1. A single portable handheld medical diagnostic tool (PHMDT) with visual and audio receiving, storing and transmitting capabilities.

2. In claim 1, the PHMDT utilizes a multitude of attachments with connectors that enable a variety of functionalities.

3. In claim 1, the PHMDT utilizes a built-in rechargeable battery system that can be recharge or powered using an AC outlet.

4. In claim 1, the PHMDT has a built-in microprocessor(s).

5. In claim 1, the PHMDT has a built-in digital display screen to display video, audio and other sensory feeds coming from the attachments.

6. In claim 5, the PHMDT also has a built-in connector to facilitate the transfer of information to electronic systems.

7. In claim 6, the built-in connector utilizes connectivity technology, such as USB, PS/2, wireless communication systems or optical communication systems and other relevant connectivity technology.

8. In claim 5, the PHMDT allows for real-time video, audio and other sensory diagnostic and therapeutic capabilities.

9. In claim 1 & 2, the PHMDT and its attachments have compact removable memory storage system.

10. In claim 2, the PHMDT utilizes physical connectivity technology, such as USB, PS/2 or any other relevant connectivity technology.

11. In claim 10, the PHMDT attachments are power by the built-in rechargeable battery system.

12. In claim 11, the attachment power requirements are light source, video, audio and other sensory receiving units.

13. In claim 1, the PHMDT incorporates the use of all kind of materials that is able to perform the desired functionality.

14. In claim 13, the PHMDT is able to be sterilized.

15. In claim 2, the PHMDT attachments are able to be sterilized and/or protected from contamination.

16. In claim 1, the PHMDT facilitates the training and education of physicians and medical personnel by displaying and transferring relevant information to electronics systems.

17. In claim 16, the PHMDT has diagnostics and therapeutic applications by displaying and transferring relevant information to electronics systems.
